(19) United States
(12) Patent Application Publication
(10) Pub. No.: US 2009/0248078 A1
(43) Pub. Date: Oct. 1, 2009

(54) SPINAL STABILIZATION DEVICE

(75) Inventor: Jack A. Dant, St. Paul, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER SPINE, INC., Minneapolis, MN (US)

(21) Appl. No.: 12/060,446

(22) Filed: Apr. 1, 2008

(51) Int. Cl. A61B 17/58; A61B 17/56

(52) U.S. Cl. 606/246; 606/240; 606/301

(57) ABSTRACT

A spinal stabilization device providing dynamic stabilization to a region of a spinal column. The spinal stabilization device is configured to be connected between a first vertebra and a second vertebra of a spinal segment. The spinal stabilization device includes a central region engaging the spinous process of the first vertebra, a first arcuate arm extending from the central region to a first piece of fixation hardware secured to the second vertebra along an arcuate path, and a second arcuate arm extending from the central region to a second piece of fixation hardware secured to the second vertebra along an arcuate path. In some embodiments, the first and second arms are pivotably connected to the first and second pieces of fixation hardware with pivot connections.

### TECHNICAL FIELD

The disclosure is directed to a system, apparatus and method for providing stabilization to one or more vertebrae of a spinal column. More particularly, the disclosure is directed to a system, apparatus and method for providing dynamic stability or support to one or more spinal segments of a spinal column.

### BACKGROUND

The spinal column of a patient includes a plurality of vertebrae linked to one another by facet joints and an intervertebral disc located between adjacent vertebrae. The facet joints and intervertebral disc allow one vertebra to move relative to an adjacent vertebra, providing the spinal column a range of motion. Diseased, degenerated, damaged, or otherwise impaired facet joints and/or intervertebral discs may cause the patient to experience pain or discomfort and/or loss of motion, thus prompting surgery to alleviate the pain and/or restore motion of the spinal column.

Accordingly, there is an ongoing need to provide alternative apparatus, devices, assemblies, systems and/or methods that can function to alleviate pain or discomfort, provide stability, such as dynamic stability, and/or restore a range of motion to a spinal segment of a spinal column.

### SUMMARY

The disclosure is directed to apparatus and methods for providing dynamic stability or support to one or more spinal segments of a spinal column.

Accordingly, one illustrative embodiment is a spinal stabilization device including an intervertebral member configured to be connected between a first vertebra and a second vertebra of a spinal column. The intervertebral member includes a central region engaged with a spinous process of the first vertebra, a first arm extending from the central region to a first pedicle of the second vertebra, and a second arm extending from the central region to a second pedicle of the second vertebra. A first fastener is secured to the first pedicle of the second vertebra, and a second fastener is secured to the second pedicle of the second vertebra. A first end of the first arm of the intervertebral member is pivotably connected to the first fastener with a first pivot connection, and a first end of the second arm of the intervertebral member is pivotably connected to the second fastener with a second pivot connection. In some embodiments, the pivot connection allows a range of motion between the intervertebral member and the first and second fasteners while positioning the intervertebral member prior to fixedly securing the intervertebral member to the first and second fasteners. In other embodiments, the pivot connections allow post-operative range of motion between the first and second arms and the first and second fasteners, providing a range of torsional motion between the first vertebra and the second vertebra subsequent completion of installation of the device.

Another illustrative embodiment is a spinal stabilization device providing dynamic stabilization to a region of a spinal column. The spinal stabilization device is configured to be connected between a first vertebra and a second vertebra of a spinal segment. The spinal stabilization device includes a central region configured to engage the spinous process of the first vertebra, a first arcuate arm extending from the central region to a first piece of fixation hardware configured to be secured to the first pedicle of the second vertebra along an arcuate path, and a second arcuate arm extending from the central region to a second piece of fixation hardware configured to be secured to the second pedicle of the second vertebra along an arcuate path. The arcuate path of the first arcuate arm extends superior to the midplane of the spinous process of the first vertebra, and the arcuate path of the second arcuate arm extends superior to the midplane of the spinous process of the first vertebra. The first arcuate arm and the second arcuate arm are formed of a resilient material allowing elastic deformation of the first and second arcuate arms to limit extension and/or flexion of the spinal column.

Yet another illustrative embodiment is a dynamic stabilization device providing dynamic stabilization to a region of a spinal column. The dynamic stabilization device is configured to be connected between a first vertebra and a second vertebra of a spinal segment. The dynamic stabilization device includes a spinous process engaging portion for engaging the spinous process of the first vertebra, a first arcuate arm extending from the spinous process engaging portion to a first fastener secured to a first anatomical region of the second vertebra along an arcuate path, and a second arcuate arm extending from the spinous process engaging portion to second a fastener secured to a second anatomical region of the second vertebra along an arcuate path. From the spinous process, the arcuate path of the first arcuate arm extends superiorly and laterally to an extent which is superior to the midplane of the spinous process of the first vertebra and lateral to the spinous process, and then the first arcuate arm extends inferiorly to the first fastener. Furthermore, from the spinous process, the arcuate path of the second arcuate arm extends superiorly and contralaterally to an extent superior to the midplane of the spinous process of the first vertebra and contralateral to the spinous process, and then the second arcuate arm extends inferiorly to the second fastener. The dynamic stabilization device may have a generally M-shape. The first arcuate arm and the second arcuate arm are formed of a resilient material allowing elastic deformation of the first and second arcuate arms to limit lateral bending, torsion, extension and/or flexion of the spinal column.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a posterior view of a spinal segment of a patient's lumbar spine with a spinal stabilization device installed thereon;

FIG. 2 is a lateral view of a spinal segment of a patient's lumbar spine with a spinal stabilization device installed thereon;

FIG. 3 is a perspective exploded view of an exemplary pivot connection between a fastener and an arm of the spinal stabilization device of FIGS. 1 and 2;

FIG. 4 is a cross-sectional view of the pivot connection shown in FIG. 3;

FIG. 5 illustrates an alternative arrangement of a spinal stabilization device installed on a portion of a spinal column;

FIG. 6 is a perspective view of a multi-level spinal fixation system including a spinal stabilization device for implantation on a patient's spinal column; and

FIG. 7 is a posterior view of a spinal segment of a patient's lumbar spine with the multi-level spinal fixation system shown in FIG. 6 installed thereon.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

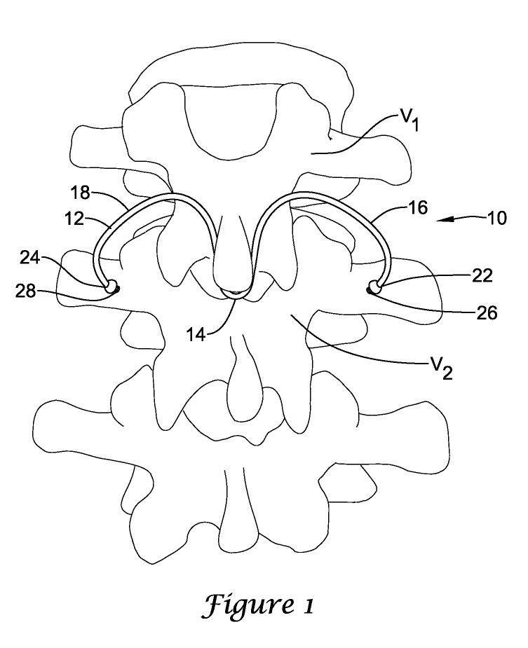

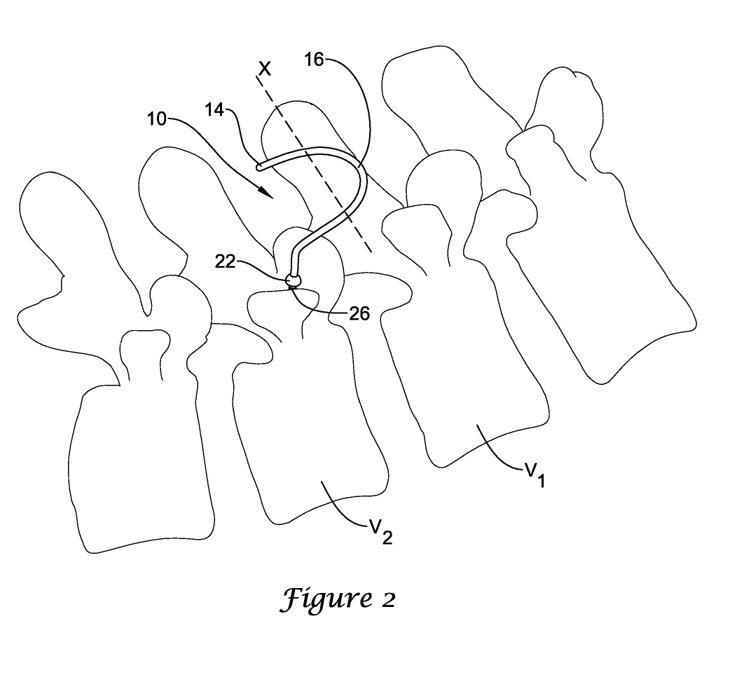

Now referring to FIGS. 1 and 2, there is shown a spinal stabilization device 10 configured to provide stabilization, such as dynamic stabilization, to a spinal segment of a spinal column. As used herein, a spinal segment is intended to refer to two or more vertebrae, the intervertebral disc(s) between the vertebrae and other anatomical elements between the vertebrae. For example, a spinal segment may include first and second adjacent vertebrae and the intervertebral disc located between the first and second vertebrae. The spinal stabilization device 10 may offer support to the spinal segment in torsion, lateral bending, extension and/or flexion. In some embodiments, the spinal stabilization device 10 may help preserve the facet joints between adjacent vertebrae by providing facet offloading and/or may stabilize or reverse neural foraminal narrowing of the spinal column.

In some embodiments, the spinal stabilization device 10 may be used to treat discogenic low back pain, degenerative spinal stenosis, disc herniations, facet syndrome, posterior element instability, adjacent level syndrome associated with spinal fusion, and/or other maladies associated with the spinal column.

The spinal stabilization device 10 may include an intervertebral member 12 configured to be connected between a first vertebra V₁ and a second vertebra V₂. As used herein, the term “intervertebral member” is intended to mean a device which extends between one vertebra to a second vertebra of a vertebral column. For example, the intervertebral member 12 may extend from the first vertebra V₁ to the second vertebra V₂ on a posterior side of the vertebral column. Although the spinal stabilization device 10 is shown being used in the lumbar region of a spinal column, it is noted that the device 10 may be used, adapted for, or modified for use in other regions of the spinal column, such as the cervical region or the thoracic region. As shown in the figures, the first vertebra V₁ is adjacent to and superior to the second vertebra V₂. For example, in some embodiments the first vertebra V₁ may be the L-4 vertebra and the second vertebra V₂ may be the L-5 vertebra. In other embodiments the first vertebra V₁ may be the L-3 vertebra and the second vertebra V₂ may be the L-4 vertebra. However, in some embodiments the first vertebra V₁ may be superior to, but not adjacent to the second vertebra V₂, or the first vertebra V₁ may be inferior to and/or adjacent or not adjacent to the second vertebra V₂. Thus, the intervertebral member 12 may be installed such that the intervertebral member 12 skips one or more levels of the spinal column. For example, the first vertebra V₁ may be the L-3 vertebra and the second vertebra V₂ may be the L-5 vertebra, or the first vertebra V₁ may be the L-1 vertebra and the second vertebra V₂ may be the L-4 vertebra. In yet other embodiments, the intervertebral member 12 may be connected between the sacrum and one or more vertebrae of the vertebral column located superior to the sacrum.

The first vertebra V₁, as shown, includes a vertebral body, first and second transverse processes, a spinous process and first and second pedicles. Similarly, the second vertebra V₂, as shown, includes a vertebral body, first and second transverse processes, a spinous process and first and second pedicles.

The intervertebral member 12 of the spinal stabilization device 10 may include a central region 14, a first arm 16 extending from the central region 14, and a second arm 18 extending from the central region 14. In some embodiments, the first arm 16 and/or the second arm 18 may be integrally formed with the central region 14, thus forming a monolithic or unitary structure including the central region 14, the first arm 16 and the second arm 18. In other embodiments, the first arm 16 and/or the second arm 18 may be separate components connected to the central region 14. In some embodiments, the intervertebral member 12 may be an elongate rod or bar bent, shaped or formed into the shape of the intervertebral member 12.

In some embodiments, the intervertebral member 12, or portions thereof, may be formed of stainless steel, nickel-titanium alloy, shape-memory alloy, titanium, polymers (e.g., polyetheretherketone (PEEK) or polyethyleneterephthalate (PET)), or other suitable material. The material and/or size of the intervertebral member 12 may be selected to provide the intervertebral member 12 with a degree of resiliency, providing the intervertebral member 12, or portions thereof, with a desired level of springiness (e.g., elastic deformation) when subjected to an applied force. The spring constant and/or size (e.g., diameter, cross-sectional area, and/or second moment of area) of the intervertebral member 12 may be selected such that when installed with the spinal column, the intervertebral member 12 may provide a desired amount of off-loading of the spinal segment to which the intervertebral member 12 is attached. The resilient material may allow for elastic deformation of the first arm 16 and/or the second arm 18 to permit and/or limit a range of extension and/or flexion of the spinal column.

The central region 14 of the intervertebral member 12 may be configured for engagement with the spinous process of the first vertebra V₁. As shown in the figures, at least a portion of the central region 14 is engaged with the spinous process of the first vertebra V₁. For example, the central region 14 may be in contact with a caudal edge of the spinous process and/or the central region 14 may be secured to the spinous process of the first vertebra V₁. In some embodiments, the central region 14 may extend around the caudal edge of the spinous process such that the spinous process is positioned in a saddle portion of the central region 14. In some embodiments, the central region 14 of the intervertebral member 12 may extend into or through a hole or opening formed into or through the spinous process of the first vertebra V₁.

The first arm 16 may extend from the central region 14 on a lateral side of the spinous process to a desired anatomical region of the second vertebra V₂, such as a first pedicle of the second vertebra V₂, and the second arm 18 may extend from the central region 14 on a contralateral side (i.e., opposite side) of the spinous process to a desired anatomical region of the second vertebra V₂, such as a second pedicle of the second vertebra V₂. The first end 22 of the first arm 16 may be coupled to a first piece of fixation hardware installed on the second vertebra V₂ and the first end 24 of the second arm 18 may be coupled to a second piece of fixation hardware installed on the second vertebra V₂. As shown in the figures, the fixation hardware may be fasteners 26, 28 such as pedicle screws, secured to the pedicles of the second vertebra V₂. In other embodiments the fixation hardware may be an elongate rod, plate or bar secured to the second vertebra V₂ with a connector of a pedicle screw of other fastener, a laminal hook, or other hardware installed on the spinal column. In some embodiments, the fasteners 26, 28 may be inserted into another anatomical region of the second vertebra V₂, such as the vertebral body, lamina, spinous process or transverse processes of the second vertebra V₂.

The first arm 16 of the intervertebral member 12 may be an arcuate arm extending along an arcuate pathway from the spinous process of the first vertebra V₁ toward and/or to the first pedicle of the second vertebra V₂. Similarly, the second arm 18 of the intervertebral member 12 may be an arcuate arm extending along an arcuate pathway from the spinous process of the first vertebra V₁ toward and/or to the second pedicle of the second vertebra V₂. In some embodiments, the arcuate pathway may be include a compound curvature including two or more distinct radii of curvature and/or directions of curvature of the first arm 16 and/or the second arm 18. As shown in FIG. 1, the arcuate pathway of the first arm 16 rises in a superior direction from the spinous process of the first vertebra V₁, then curves downward in an inferior direction toward the first pedicle of the second vertebra V₂. Similarly, the arcuate pathway of the second arm 18 rises in a superior direction from the spinous process of the first vertebra V₁, then curves downward in an inferior direction toward the second pedicle of the second vertebra V₂.

A lateral midplane X bisecting the spinous process of the first vertebra V₁ into a superior portion (e.g. superior half) and an inferior portion (e.g., inferior half) is shown in FIG. 2. The arcuate pathway through which the first arm 16 travels through, after leaving the spinous process of the first vertebra V₁, extends superior to the midplane of the spinous process of the first vertebra V₁ before the first arm 16 changes direction and extends toward the first pedicle of the second vertebra V₂. Similarly, the arcuate pathway through which the second arm 18 travels through, after leaving the spinous process of the first vertebra V₁, extends superior to the midplane of the spinous process of the first vertebra V₁ before the second arm 18 changes direction and extends toward the second pedicle of the second vertebra V₂.

As shown in FIG. 1, the pathways through which the first and second arms 16, 18 extend, may provide the intervertebral member 12 with a generally M-shape. It can be seen that from the spinous process of the first vertebra V₁, the first arcuate arm 16 extends superiorly and laterally to an upper extent which is superior to the midplane of the spinous process and lateral to the spinous process, and then extends inferiorly toward the fastener 26 secured to the first pedicle of the second vertebra V₂. Furthermore, from the spinous process of the first vertebra V₁, the second arcuate arm 18 extends superiorly and contralaterally to an upper extent which is superior to the midplane of the spinous process and contralateral to the spinous process, and then extends inferiorly toward the fastener 28 secured to the second pedicle of the second vertebra V₂. The shape of the intervertebral member 12 may provide a range of motion not achievable by other devices.

In other embodiments, the intervertebral member 12 may be installed in an opposite orientation as that shown in FIGS. 1 and 2. In other words, the first vertebra V₁ may be located inferior to the second vertebra V₂.

In such an embodiment, the central region 14 of the intervertebral member 12 may engage a cephalad edge of the spinous process of the first vertebra V₁. The first arm 16 of the intervertebral member 12 may be an arcuate arm extending along an arcuate pathway from the spinous process of the first vertebra V₁ toward and/or to the first pedicle of the second vertebra V₂. Similarly, the second arm 18 of the intervertebral member 12 may be an arcuate arm extending along an arcuate pathway from the spinous process of the first vertebra V₁ toward and/or to the second pedicle of the second vertebra V₂. In some embodiments, the arcuate pathway may be include a compound curvature including two or more distinct radii of curvature and/or directions of curvature of the first arm 16 and/or the second arm 18. Thus, the arcuate pathway of the first arm 16 may extend downward in an inferior direction from the spinous process of the first vertebra V₁, then curve upward in superior direction toward the first pedicle of the second vertebra V₂. Similarly, the arcuate pathway of the second arm 18 may extend downward in an inferior direction from the spinous process of the first vertebra V₁, then curve upward in a superior direction toward the second pedicle of the second vertebra V₂.

A lateral midplane X may bisect the spinous process of the first vertebra V₁ into a superior portion (e.g. superior half) and an inferior portion (e.g., inferior half). The arcuate pathway through which the first arm 16 travels through, after leaving the spinous process of the first vertebra V₁, extends inferior to the midplane of the spinous process of the first vertebra V₁ before the first arm 16 changes direction and extends toward the first pedicle of the second vertebra V₂. Similarly, the arcuate pathway through which the second arm 18 travels through, after leaving the spinous process of the first vertebra V₁, extends inferior to the midplane of the spinous process of the first vertebra V₁ before the second arm 18 changes direction and extends toward the second pedicle of the second vertebra V₂.

Thus, the pathways through which the first and second arms 16, 18 extend, may provide the intervertebral member 12 with a generally W-shape. It can be understood that from the spinous process of the first vertebra V₁, the first arcuate arm 16 may extend inferiorly and laterally to a lower extent which is inferior to the midplane of the spinous process and lateral to the spinous process, and then may extend superiorly toward the fastener 26 secured to the first pedicle of the second vertebra V₂. Furthermore, from the spinous process of the first vertebra V₁, the second arcuate arm 18 may extend inferiorly and contralaterally to a lower extent which is inferior to the midplane of the spinous process and contralateral to the spinous process, and then may extend superiorly toward the fastener 28 secured to the second pedicle of the second vertebra V₂. The shape of the intervertebral member 12 may provide a range of motion not achievable by other devices.

The first end 22 of the first arm 16 may be connected to a piece of fixation hardware, such as a first fastener 26. The first fastener 26 may be a pedicle screw secured to a first pedicle of the second vertebra V₂. For instance, the fastener 26 may include a threaded portion screwed into the first pedicle of the second vertebra V₂ and a head portion to which the first arm 16 is connected to. Additionally, the first end 24 of the second arm 18 may be connected to a piece of fixation hardware, such as a second fastener 28. The second fastener 28 may be a pedicle screw secured to a second pedicle of the second vertebra V₂. For instance, the fastener 28 may include a threaded portion screwed into the second pedicle of the second vertebra V₂ and a head portion to which the second arm 18 is connected to.

Again referring to FIGS. 1 and 2, in some embodiments, the first arm 16 may be rigidly connected to the fastener 26 such that when installed, the first arm 16 is unable to move, thus fixed, relative to the fastener 26. In some embodiments the first end 22 of the first arm 16 may be pivotably connected to the fastener 26 with a pivot connection, providing a range of motion between the first arm 16 and the first fastener 26 while positioning the intervertebral member 12 prior to fixedly securing the intervertebral member 12 to the first fastener 26. For example, in some embodiments the first end 22 of the first arm 16 may be coupled to a polyaxial screw similar to the polyaxial connection disclosed in U.S. Pat. App. Pub. No. 2006/0052786, incorporated herein by reference. Such a pivot connection may provide a range of motion between the first arm 16 and the first fastener 26 during installation in order to properly position the intervertebral member 12. Once the proper position of the intervertebral member 12 is attained, final fixed securement of the first arm 16 to the first fastener 26 may be accomplished by tightening a nut onto a threaded stud of the first fastener 26, for example, to fixedly secure the first arm 16 to the first fastener 26. In other embodiments the first arm 16 may be connected to the fastener 26 with a pivot connection such that when installed the first arm 16 may be able to move relative to the fastener 26, providing a range of motion between the first arm 16 and the fastener 26. In other words, the pivot connection between the first end 22 of the first arm 16 and the first fastener 26 may be maintained post-operatively. Such a pivot connection may provide a range of torsional motion between the first vertebra V₁ and the second vertebra V₂ with the intervertebral member 12 attached to the spinal segment subsequent to completion of installation of the device.

Similarly, in some embodiments, the second arm 18 may be rigidly connected to the fastener 28 such that when installed, the second arm 18 is unable to move, thus fixed, relative to the fastener 28. In some embodiments the first end 24 of the second arm 18 may be pivotably connected to the fastener 28 with a pivot connection, providing a range of motion between the second arm 18 and the second fastener 28 while positioning the intervertebral member 12 prior to fixedly securing the intervertebral member 12 to the second fastener 28. For example, in some embodiments the first end 24 of the second arm 18 may be coupled to a polyaxial screw similar to the polyaxial connection disclosed in U.S. Pat. App. Pub. No. 2006/0052786, incorporated herein by reference. Such a pivot connection may provide a range of motion between the second arm 18 and the second fastener 28 during installation in order to properly position the intervertebral member 12. Once the proper position of the intervertebral member 12 is attained, final fixed securement of the second arm 18 to the second fastener 28 may be accomplished by tightening a nut onto a threaded stud of the second fastener 28, for example, to fixedly secure the second arm 18 to the second fastener 28. In other embodiments the second arm 18 may be connected to the fastener 28 with a pivot connection such that when installed the second arm 18 may be able to move relative to the fastener 28, providing a range of motion between the second arm 18 and the fastener 28. In other words, the pivot connection between the first end 24 of the second arm 18 and the second fastener 28 may be maintained post-operatively. Such a pivot connection may provide a range of torsional motion between the first vertebra V₁ and the second vertebra V₂ with the intervertebral member 12 attached to the spinal segment subsequent to completion of installation of the device.

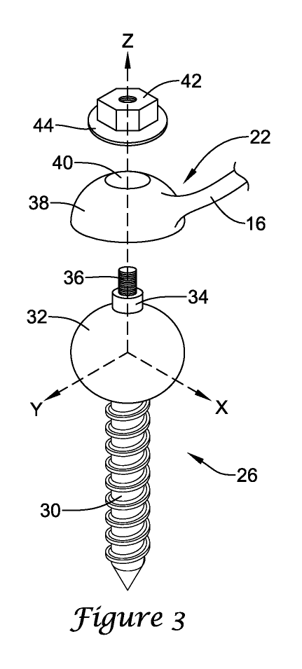

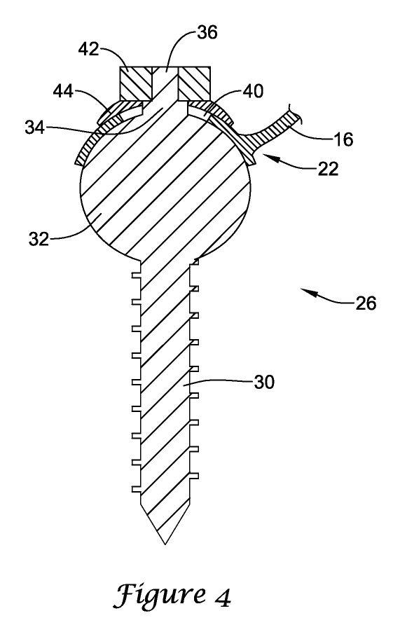

One exemplary pivot connection between an arm 16, 18 of the intervertebral member 12 and a fastener 26, 28 is shown in FIGS. 3 and 4. Although the pivot connection between the first arm 16 and the first fastener 26 is shown in FIGS. 3 and 4, it is noted that the pivot connection between the second arm 18 and the second fastener 28 may be substantially similar to that shown in the figures. In the interest of brevity, the construction, arrangement and operation of the pivot connection between the second arm 18 and the second fastener 28 will not be reiterated.

The pivot connection may be a ball-and-socket connection, or other connection providing the arm 16 with a range of motion relative to the fastener 26. For example, the fastener 26 may include a threaded portion 30 for securing the fastener 26 to a bone, such as the pedicle of a vertebra, and a ball 32 (e.g., a spherical or semi-spherical portion of a ball-and-socket joint) including a convex surface. A post 34, which may include a threaded portion 36, may extend from the ball 32.

The first end 22 of the first arm 16 may include a socket 38 (e.g., a bowl-shaped portion of a ball-and-socket joint) including a concave surface. The socket 38 may include an opening 40 through which the post 34 may extend through. As shown in FIG. 4, the opening 40 may be sized larger than the post 34 such that the post 34 may move within the opening 40 as the first arm 16 is pivoted relative to the fastener 26. A nut 42 may be provided to threadedly engage the threads of the post 34 in order to secure the first arm 16 to the fastener 26. Additionally, a washer 44, such as a nylon washer, may be positioned between the nut 42 and the socket 38 to span the opening 40 and/or reduce friction at the pivot connection. In some embodiments, the washer 44 may be a component of the nut 42, such as a flanged edge of the nut 42.

When assembled, the ball 32 may be disposed in the socket 38 such that the concave surface of the socket 38 faces and/or contacts the convex surface of the ball 32. The interface between the ball 32 and the socket 38 may have a low coefficient of friction, allowing for movement between the ball 32 and socket 38 such that the ball 32 may rotate in the socket 38. In some embodiments, the ball-and-socket joint allows for rotary motion of the first arm 16 relative to the first fastener 26 in all directions (i.e., yaw, pitch and roll), or one or more of yaw, pitch and/or roll motions. As used herein, roll is intended to describe rotational movement of the socket 38 relative to the ball 32 about the X-axis shown in FIG. 3, pitch is intended to describe rotational movement of the socket 38 relative to the ball 32 about the Y-axis shown in FIG. 3, and yaw is intended to describe rotational movement of the socket 38 relative to the ball 32 about the Z-axis shown in FIG. 3.

In the XYZ coordinate system shown in FIG. 3, the Z-axis lies along the longitudinal axis of the threaded portion 30 of the fastener 26 which extends in a generally posterior direction when the fastener 26 is installed on a patient's spinal column, the X-axis, which is perpendicular to the Z-axis, extends in a generally superior direction when the fastener 26 is installed on a patient's spinal column (which may be generally in the direction in which the arm 16 extends from the socket 38), and the Y-axis, which is perpendicular to both the X-axis and the Z-axis, extends in a generally lateral direction when the fastener 26 is installed on a patient's spinal column.

When fully assembled in a patient's body, pivotable motion between the ball 32 of the fastener 26 and the socket 38 of the arm 16 may be maintained even with the components, such as with the nut 42 fully tightened onto the threaded portion 36 of the post 34, or other fastening element fully tightened. Thus, tightening of the nut 42, or other fastening element, does not lock the socket 38 onto the ball 32 in a singular fixed orientation, but rather prevents the socket 38 from dissociating from the ball 32 while maintaining pivotable movement between the ball 32 and socket 38. For example, the nut 42 may be a self-locking nut and/or the nut 42 may “bottom-out” on the threaded portion 36 of the post 34 without applying sufficient force to the socket 38 to prevent the socket 38 from moving relative to the ball 32. Thus, pivotable movement at the pivot connection may be retained once the spinal stabilization device 10 is installed on a spinal segment of a patient's spinal column.

In other embodiments the first end 22 of the first arm 16 may be pivotably connected to the fastener 26 with a pivot connection, providing a range of motion between the first arm 16 and the first fastener 26 while positioning the intervertebral member 12 prior to fixedly securing the intervertebral member 12 to the first fastener 26. Such a pivot connection may provide a range of motion between the first arm 16 and the first fastener 26 during installation in order to properly position the intervertebral member 12. Once the proper position of the intervertebral member 12 is attained, final fixed securement of the first arm 16 to the first fastener 26 may be accomplished by tightening a nut onto a threaded stud of the first fastener 26, for example, to fixedly secure the first arm 16 to the first fastener 26 in a singular fixed orientation.

The fastener 26 may be formed of any desired material, such as stainless steel, titanium, tantalum, or other desired material. In some embodiments, the fastener 26, or the threaded portion 30 thereof, may be formed of a porous metal, such as Trabecular Metal™ (a tantalum open cell material sold by Zimmer Trabecular Metal Technology, Parsippany, N.J.), which allows bony ingrowth in the pores of the metal. Bony ingrowth in the pores of the Trabecular Metal™ may help prevent the threaded portion 30 of the fastener 26 from backing out, unscrewing, or working loose, from the movement between the arm 16 and the fastener 26 at the pivot connection when the spinal stabilization device 10 is implanted.

An exemplary installation procedure for installing the spinal stabilization device 10 will now be described. Installation of the spinal stabilization device 10 may be performed by initially accessing the desired spinal segment to which the spinal stabilization device 10 will be installed. Access may be gained via a midline or Wiltse approach, for example. In some embodiments, an access device, such as an expandable cannula may be used to obtain access to the desired spinal segment. With the spinal segment exposed, the fasteners 26, 28 (e.g., pedicle screws) may be installed in the first and second pedicles of the second vertebra V₂.

Next, as necessary, the medical personnel may bluntly dissect tissue to access the spinous process of the first vertebra V₁ and associated ligaments. The interspinous ligament may be pierced at a desired location, such as just inferior to the spinous process of the first vertebra, forming an opening through the interspinous ligament. It is noted that installation of the spinal stabilization device 10 may not require removal of the supraspinous ligament, and may only require piercing the interspinous ligament, largely maintaining the patient's posterior ligamentous complex in place.

Using anterior/posterior (A/P) and lateral fluoroscopy, the medical personnel may determine the appropriate size of spinal stabilization device 10 to be installed in the patient. Once the appropriate sized spinal stabilization device 10 has been determined and selected, the spinal stabilization device 10 may be inserted into the cavity dissected around the spinal segment. For example, the second arm 18 of the intervertebral member 12 may be inserted through the opening formed through the interspinous ligament until the central region 14 of the intervertebral member 12 is positioned through the opening through the interspinous ligament at the spinous process of the first vertebra V₁. The central region 14 may be engaged with the spinous process of the first vertebra V₁. For example, the central region 14 of the intervertebral member 12 may be placed or rested against the caudal edge of the spinous process of the first vertebra V₁, and/or the central region 14 may be secured to the spinous process.

With the intervertebral member 12 positioned through the opening through the interspinous ligament, the first end 22 of the first arm 16 may be connected to the first fastener 26 and the first end 24 of the second arm 18 may be connected to the second fastener 28. For instance, the sockets 38 at the ends of the first and second arms 16, 18 may be placed over the balls 32 of the fasteners 26, 28 and coupled together. For example, the nuts 42 may be threaded on the threaded portion 36 of the posts 34, securing the sockets 38 onto the balls 32 of the ball-and-socket pivot connections. In other embodiments, another fastener may be tightened to secure the sockets 38 onto the balls 32 of the ball-and-socket pivot connections. When fully assembled in a patient's body, pivotable motion between the ball 32 of the fastener 26, 28 and the socket 38 of the arm 16, 18 may be maintained even with the components, such as with the nut 42 fully tightened onto the threaded portion 36 of the post 34. Thus, tightening of the nut 42, or other fastener, does not lock the socket 38 onto the ball 32 in a singular fixed orientation, but rather prevents the socket 38 from dissociating from the ball 32 while maintaining pivotable movement between the ball 32 and socket 38.

If necessary or desirable, the central region 14 of the intervertebral member 12 may be secured to the spinous process of the first vertebra V₁ prior to, or after the arms 16, 18 of the intervertebral member 12 are connected to the fasteners 26, 28.

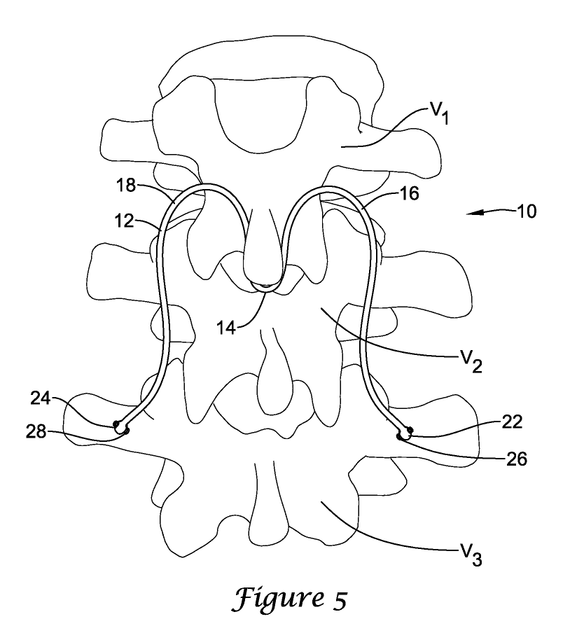

FIG. 5 illustrates an alternative arrangement of the spinal stabilization device 10. As shown in FIG. 5, the spinal stabilization device 10 may be configured to skip (e.g., pass over, extend across) one or more levels of the spinal column. For example, as shown, the intervertebral member 12 may be configured to be connected between a first vertebra V₁ and a third vertebra V₃, with one or more vertebrae, such a second vertebra V₂ located between the first vertebra V₁ and the third vertebra V₃. In the embodiment shown in FIG. 5, the intervertebral member 12 passes posterior to the second vertebra V₂, but is not secured to the second vertebra V₂.

The central region 14 of the intervertebral member 12 may be configured for engagement with the spinous process of the first vertebra V₁. As shown in FIG. 5, at least a portion of the central region 14 is engaged with the spinous process of the first vertebra V₁. For example, the central region 14 may be in contact with a caudal edge of the spinous process and/or the central region 14 may be secured to the spinous process of the first vertebra V₁. In some embodiments, the central region 14 may extend around the caudal edge of the spinous process such that the spinous process is positioned in a saddle portion of the central region 14. In some embodiments, the central region 14 of the intervertebral member 12 may extend into or through a hole or opening formed into or through the spinous process of the first vertebra V₁.

The first arm 16 may extend from the central region 14 on a lateral side of the spinous process to a desired anatomical region of the third vertebra V₃, such as a first pedicle of the third vertebra V₃, and the second arm 18 may extend from the central region 14 on a contralateral side (i.e., opposite side) of the spinous process to a desired anatomical region of the third vertebra V₃, such as a second pedicle of the third vertebra V₃. The first end 22 of the first arm 16 may be coupled to a first piece of fixation hardware installed on the third vertebra V₃ and the first end 24 of the second arm 18 may be coupled to a second piece of fixation hardware installed on the third vertebra V₃. As shown in FIG. 5, the fixation hardware may be fasteners 26, 28 such as pedicle screws, secured to the pedicles of the third vertebra V₃. In other embodiments the fixation hardware may be an elongate rod, plate or bar secured to the third vertebra V₃ with a connector of a pedicle screw of other fastener, a laminal hook, or other hardware installed on the spinal column. In some embodiments, the fasteners 26, 28 may be inserted into another anatomical region of the third vertebra V₃, such as the vertebral body, lamina, spinous process or transverse processes of the third vertebra V₃.

The first arm 16 of the intervertebral member 12 may be an arcuate arm extending along an arcuate pathway from the spinous process of the first vertebra V₁ toward and/or to the first pedicle of the third vertebra V₃. As shown in FIG. 5, the arcuate pathway may follow a compound curvature, providing the first arm 16 of the intervertebral member 12 with a compound curvature having two or more radii of curvature and/or directions of curvature. For example as shown, from the spinous process of the first vertebra V₁, the first arm 16 may curve laterally outward and superiorly, then curve inferiorly while curving laterally inward toward the sagittal plane (i.e., vertical midplane), and then curve laterally outward and/or anteriorly toward the first fastener 26 secured to the third vertebra V₃. It is understood however, that in other embodiments, the arcuate pathway may include other configurations of compound curvatures as desired.

Similarly, the second arm 18 of the intervertebral member 12 may be an arcuate arm extending along an arcuate pathway from the spinous process of the first vertebra V₁ toward and/or to the second pedicle of the third vertebra V₃. As shown in FIG. 5, the arcuate pathway may follow a compound curvature, providing the second arm 18 of the intervertebral member 12 with a compound curvature having two or more radii of curvature and/or directions of curvature. For example as shown, from the spinous process of the first vertebra V₁, the second arm 18 may curve laterally outward and superiorly, then curve inferiorly while curving laterally inward toward the sagittal plane (i.e., vertical midplane), and then curve laterally outward and/or anteriorly toward the second fastener 28 secured to the third vertebra V₃. It is understood however, that in other embodiments, the arcuate pathway may include other configurations of compound curvatures as desired.

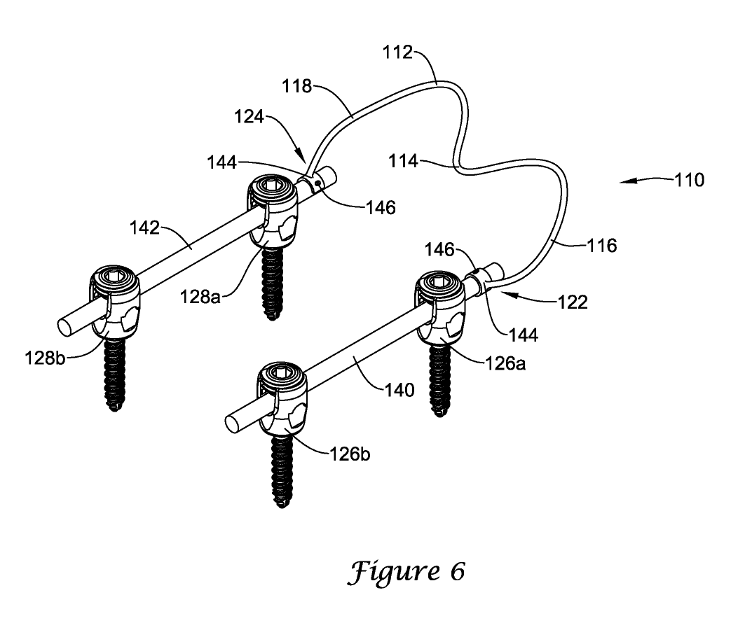

Another embodiment of a spinal stabilization device 110, similar to the spinal stabilization device 10, is illustrated in FIG. 6. The spinal stabilization device 110 may include an intervertebral member 112 configured to be connected between a first vertebra and a second vertebra.

The intervertebral member 112 of the spinal stabilization device 110 may include a central region 114, a first arm 116 extending from the central region 114, and a second arm 118 extending from the central region 114. In some embodiments, the first arm 116 and/or the second arm 118 may be integrally formed with the central region 114, thus forming a monolithic or unitary structure including the central region 114, the first arm 116 and the second arm 118. In other embodiments, the first arm 116 and/or the second arm 118 may be separate components connected to the central region 114. In some embodiments, the intervertebral member 112 may be an elongate rod or bar bent, shaped or formed into the shape of the intervertebral member 112.

In some embodiments, the intervertebral member 112, or portions thereof, may be formed of stainless steel, nickel-titanium alloy, shape-memory alloy, titanium, polymers (e.g., polyetheretherketone (PEEK) or polyethyleneterephthalate (PET)), or other suitable material. The material and/or size of the intervertebral member 112 may be selected to provide the intervertebral member 112 with a degree of resiliency, providing the intervertebral member 112, or portions thereof, with a desired level of springiness (e.g., elastic deformation) when subjected to an applied force. The spring constant and/or size (e.g., diameter, cross-sectional area, and/or second moment of area) of the intervertebral member 112 may be selected such that when installed with the spinal column, the intervertebral member 112 may provide a desired amount of off-loading of the spinal segment to which the intervertebral member 112 is attached. The resilient material may allow for elastic deformation of the first arm 116 and/or the second arm 118 to permit and/or limit a range of extension and/or flexion of the spinal column.

The central region 114 of the intervertebral member 112 may be configured for engagement with the spinous process of the first vertebra. For example, the central region 114 may be configured to contact with a caudal edge of the spinous process and/or the central region may be secured to the spinous process of the first vertebra. In some embodiments, the central region 114 may be configured to extend around the caudal edge of the spinous process such that the spinous process is positioned in a saddle portion of the central region 114. In some embodiments, the central region 114 of the intervertebral member 112 may extend into or through a hole or opening formed into or through the spinous process of the first vertebra.

The first arm 116 may extend from the central region 114 on a lateral side of the spinous process to a desired anatomical region of a second vertebra, and the second arm 118 may extend from the central region 114 on a contralateral side (i.e., opposite side) of the spinous process to a desired anatomical region of a second vertebra. For instance, the first arm 116 of the intervertebral member 112 may be an arcuate arm extending along an arcuate pathway from the spinous process of the first vertebra toward an anatomical region of the second vertebra, and the second arm 118 of the intervertebral member 112 may be an arcuate arm extending along an arcuate pathway from the spinous process of the first vertebra toward an anatomical region of the second vertebra. The arcuate pathway of the first arm 16 and the arcuate pathway of the second arm 118 may be substantially similar to the pathways of the arms 16, 18 of the intervertebral member 12 described above. For instance, the pathways through which the first and second arms 116, 118 extend, may provide the intervertebral member 112 with a generally M-shape. Thus, in the interest of brevity, the pathways of the first arm 116 and second arm 118 and their association with anatomical regions of the spinal segment will not be repeated.

The first end 122 of the first arm 116 may be connected to a piece of fixation hardware, such as a first elongate rod 140, or a plate, secured to one or more vertebrae of the patient's spinal column. Additionally, the first end 124 of the second arm 118 may be connected to a piece of fixation hardware, such as a second elongate rod 142, or a plate, secured to one or more vertebrae of the patient's spinal column. For instance, the first and second elongate rods 140, 142 may be secured to two or more vertebrae on opposing sides of the midline of the spinal column with fasteners 126, 128, such as pedicle screws including a connector connected to the elongate rods 140, 142. One example of a pedicle screw which may be coupled to an elongate rod for implanting into a vertebra of a patient is disclosed in U.S. Pat. No. 7,144,396, incorporated herein by reference.

For example, the first end 122 of the first arm 116 and the first end 124 of the second arm 118 may each include a saddle portion 144 configured to extend around a portion of the perimeter (e.g., circumference) of the rod 140, 142. The saddle portion 144 may include a set screw 146, or other fastening element, which may be tightened to secure the saddle portion 144 of the arm 116, 118 to the rod 140, 142.

The multi-level spinal fixation system 180 shown in FIG. 6, which includes the spinal stabilization device 110 and the fixation hardware including the elongate rods 140, 142 and fasteners 126, 128 may be installed on a spinal segment of a patient's spinal column to treat, reduce, delay, and/or prevent adjacent level degenerative disc disease, for example. It is contemplated that the spinal stabilization device 110 may be installed as an add-on to an existing (e.g., previously installed) spinal fixation apparatus including fasteners and rods secured to two or more vertebrae, or the spinal stabilization device 110 may be used in a new, concurrently installed multi-level spinal fixation system including fasteners and rods secured to two or more vertebrae.

The central region 114 of the intervertebral member 112 may be engaged with the spinous process of a first vertebra, the first fasteners 126a, 128a may be secured to desired regions, such as the pedicles, of a second vertebra, and the second fasteners 126b, 128b may be secured to desired regions, such as the pedicles, of a third vertebra. For instance, the first vertebra may be adjacent and/or superior to the second vertebra, which may be adjacent and/or superior to the third vertebra. For instance, in some embodiments the first vertebra may be the L-3 vertebra, the second vertebra may be the L-4 vertebra, and the third vertebra may be the L-5 vertebra, for example.

If the spinal stabilization device 110 is being installed on an existing spinal fixation apparatus, it may be necessary to install the saddle portion 144 of the arms 116, 118 on a portion of the rod 140, 142 between the fastener 126a, 128a secured to one vertebra and the fastener 126b, 128b secured to an adjacent vertebra. If the spinal stabilization device 110 is being installed concurrently with rods 140, 142 and fasteners 126, 128 of a multi-level spinal fixation system, it may be possible to install the saddle portion 144 of the arms 116, 118 on a portion of the rod 140, 142 extending beyond (e.g., superior to) the fasteners 126a, 128a as shown in FIG. 5. In other embodiments, the saddle portion 144 of the arms 116, 118 may be installed on a portion of the rod 140, 142 located between the fasteners 126a, 128a and fasteners 126b, 128b.

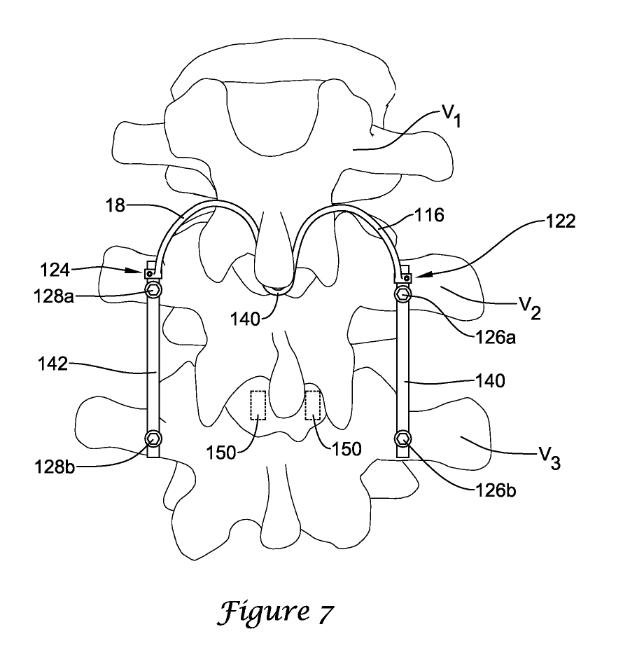

FIG. 7 illustrates the multi-level spinal fixation system 180 including the spinal stabilization device 110 installed on a spinal segment of a patient's spinal column. As shown in FIG. 7, the central region 114 of the intervertebral member 112 is engaged with the spinous process of a first vertebra V₁. For example, the central region 114 may be in contact with a caudal edge of the spinous process and/or the central region 114 may be secured to the spinous process of the first vertebra V₁. In some embodiments, the central region 114 may extend around the caudal edge of the spinous process such that the spinous process is positioned in a saddle portion of the central region 114. In some embodiments, the central region 114 of the intervertebral member 12 may extend into or through a hole or opening formed into or through the spinous process of the first vertebra V₁.

The first elongate rod 140 may be secured to the second vertebra V₂ and the third vertebra V₃ with fasteners 126a, 126b secured to the second vertebra V₂ and the third vertebra V₃, respectively. Similarly, the second elongate rod 142 may be secured to the second vertebra V₂ and the third vertebra V₃ with fasteners 128a, 128b secured to the second vertebra V₂ and the third vertebra V₃, respectively. The first end 122 of the first arm 116 of the intervertebral member 112 may be secured to the first elongate rod 140 proximate the first fastener 126a, and the first end 124 of the second arm 118 of the intervertebral member 112 may be secured to the second elongate rod 142 proximate the first fastener 128a.

The fixation hardware, including the elongate rods 140, 142 and the fasteners 126, 128 may restore the alignment, support and/or stabilize the position of the second vertebra V₂ relative to the third vertebra V₃. For example, the fixation hardware may limit the range of motion between the second vertebra V₂ and the third vertebra V₃. In some embodiments one or more implants 150, such as prosthetic discs, fusion devices or spacers, may be inserted in the disc space (e.g., interdiscal space) between the second vertebra V₂ and third vertebra V₃ to promote fusion of the second vertebra V₂ to the third vertebra V₃, replace or restore an intervertebral disc, and/or provide desired spacing between the second vertebra V₂ and the third vertebra V₃.

The inclusion of the spinal stabilization device 110 may treat, reduce, delay, and/or prevent adjacent level syndrome associated with spinal fusion of the second vertebra V₂ to the third vertebra V₃ and/or other maladies associated with the spinal column. For instance, the spinal stabilization device 110 may provide stabilization, such as dynamic stabilization, between the first vertebra V₁ and the second vertebra V₂. For example, the spinal stabilization device 110 may offer support between the first vertebra V₁ and the second vertebra V₂, allowing a degree of extension, torsion, lateral bending and/or flexion between the first vertebra V₁ and the second vertebra V₂. In some embodiments, the spinal stabilization device 110 may help preserve the facet joints between the first vertebra V₁ and the second vertebra V₂ by providing facet offloading and/or may stabilize or reverse neural foraminal narrowing of the spinal column.

The foregoing methods, apparatus, and systems may provide treatment to a variety of spine conditions in a manner that preserves a degree of motion between adjacent vertebrae of a spinal segment of a patient's spinal column. Accordingly, the patient may experience a more normal range of motion of the spine and/or a reduction in the occurrences of additional procedures to treat/prevent maladies of the spinal column.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A spinal stabilization device, comprising:
a member configured to be connected between a first bone member and a second bone member of a spinal column;
the member including a central region engaged with a spinous process of the first bone member, a first arm extending from the central region to a first anatomical region of the second bone member, and a second arm extending from the central region to a second anatomical region of the second bone member;
a first fastener secured to the first anatomical region of the second bone member;
a second fastener secured to the second anatomical region of the second bone member;
a first end of the first arm of the member pivotably connected to the first fastener with a first pivot connection, wherein the first pivot connection allows a range of motion between the first arm and the first fastener, providing a range of torsional motion between the first bone member and the second bone member; and
a first end of the second arm of the member pivotably connected to the second fastener with a second pivot connection, wherein the second pivot connection allows a range of motion between the second arm and the second fastener, providing a range of torsional motion between the first bone member and the second bone member.

2. The spinal stabilization device of claim 1, wherein the first arm follows an arcuate pathway from the spinous process of the first bone member to the first anatomical region of the second bone member; and
wherein the second arm follows an arcuate pathway from the spinous process of the first bone member to the second anatomical region of the second bone member.

3. The spinal stabilization device of claim 2, wherein the arcuate pathway of the first arm rises in a superior direction from the spinous process of the first bone member, then curves downward in an inferior direction toward the first anatomical region of the second bone member; and
wherein the arcuate pathway of the second arm rises in a superior direction from the spinous process of the first bone member, then curves downward in an inferior direction toward the second anatomical region of the second bone member.

4. The spinal stabilization device of claim 1, wherein the first pivot connection is a first ball-and-socket joint including a ball having a convex surface disposed within a socket having a concave surface; and
the second pivot connection is a second ball-and-socket joint including a ball having a convex surface disposed within a socket having a concave surface.

5. The spinal stabilization device of claim 4, wherein the first ball-and-socket joint allows for rotary motion of the first arm relative to the first fastener in all directions; and
wherein the second ball-and-socket joint allows for rotary motion of the second arm relative to the second fastener in all directions.

6. The spinal stabilization device of claim 4, wherein the ball of the first pivot connection is a portion of the first fastener, and wherein the first end of the first arm includes the socket of the first pivot connection.

7. The spinal stabilization device of claim 6, wherein the ball of the second pivot connection is a portion of the second fastener, and wherein the first end of the second arm includes the socket of the second pivot connection.

8. The spinal stabilization device of claim 1, wherein the member is M-shaped.

9. The spinal stabilization device of claim 1, wherein the first arm and the second arm are formed of a resilient material allowing elastic deformation of the first and second arms to limit extension and/or flexion of the spinal column.

10. The spinal stabilization device of claim 1, wherein the first bone member is superior to the second bone member.

11. The spinal stabilization device of claim 1, wherein the first bone member is inferior to the second bone member.

12. A spinal stabilization device providing dynamic stabilization to a region of a spinal column, the spinal stabilization device configured to be connected between a first vertebra and a second vertebra, each of the first and second vertebrae including a vertebral body, first and second pedicles, and a spinous process, the first vertebra located superior to the second vertebra, wherein an imaginary lateral midplane bisects the spinous process of the first vertebra into a superior portion and a inferior portion, the spinal stabilization device comprising:
a central region engaging the spinous process of the first vertebra;
a first arcuate arm extending from the central region to a first piece of fixation hardware secured to the first pedicle of the second vertebra along an arcuate path, wherein the arcuate path of the first arcuate arm extends superior to the midplane of the spinous process of the first vertebra;
a second arcuate arm extending from the central region to a second piece of fixation hardware secured to the second pedicle of the second vertebra along an arcuate path, wherein the arcuate path of the second arcuate arm extends superior to the midplane of the spinous process of the first vertebra;
wherein the first arcuate arm and the second arcuate arm are formed of a resilient material allowing elastic deformation of the first and second arcuate arms to limit extension and/or flexion of the spinal column.

13. The spinal stabilization device of claim 12, wherein the central region of the spinal stabilization device rests against a caudal edge of the spinous process of the first vertebra.

14. The spinal stabilization device of claim 12, wherein the central region of the spinal stabilization device is secured to the spinous process of the first vertebra.

15. The spinal stabilization device of claim 12, wherein the first piece of fixation hardware is a fastener engaged with the first pedicle and the second piece of fixation hardware is a fastener engaged with the second pedicle.

16. The spinal stabilization device of claim 12, wherein the first piece of fixation hardware is an elongate rod attached to the first pedicle with a pedicle screw, and the second piece of fixation hardware is an elongate rod attached to the second pedicle with a pedicle screw.

17. The spinal stabilization device of claim 12, wherein the first arcuate arm is pivotably connected to the first piece of fixation hardware with a first pivot connection, and wherein the second arcuate arm is pivotably connected to the second piece of fixation hardware with a second pivot connection.

18. The spinal stabilization device of claim 17, wherein the first pivot connection is a ball-and-socket joint; and
wherein the second pivot connection is a ball-and-socket joint.

19. The spinal stabilization device of claim 12, wherein the arcuate path of the first arcuate arm includes a compound curvature; and
wherein the arcuate path of the second arcuate arm includes a compound curvature.

20. A dynamic stabilization device providing dynamic stabilization to a region of a spinal column, the dynamic stabilization device configured to be connected between a first vertebra and a second vertebra, each of the first and second vertebrae including a vertebral body, first and second pedicles, and a spinous process, the first vertebra located superior to the second vertebra, wherein an imaginary lateral midplane bisects the spinous process of the first vertebra into a superior portion and a inferior portion, the dynamic stabilization device comprising:
a spinous process engaging portion for engaging the spinous process of the first vertebra;
a first arcuate arm extending from the spinous process engaging portion to a first piece of fixation hardware secured to a first anatomical region of the second vertebra along an arcuate path, wherein from the spinous process the arcuate path of the first arcuate arm extends superiorly and laterally to an extent which is superior to the midplane of the spinous process of the first vertebra and lateral to the spinous process, and then the first arcuate arm extends inferiorly to the first piece of fixation hardware;
a second arcuate arm extending from the spinous process engaging portion to a second piece of fixation hardware secured to a second anatomical region of the second vertebra along an arcuate path, wherein from the spinous process the arcuate path of the second arcuate arm extends superiorly and contralaterally to an extent superior to the midplane of the spinous process of the first vertebra and contralateral to the spinous process, and then the second arcuate arm extends inferiorly to the second piece of fixation hardware; and
wherein the first arcuate arm and the second arcuate arm are formed of a resilient material allowing elastic deformation of the first and second arcuate arms to limit extension and/or flexion of the spinal column.

21. The spinal stabilization device of claim 20, wherein the dynamic stabilization device has a generally M-shape.

22. The spinal stabilization device of claim 20, wherein the first piece of fixation hardware is a first fastener, and the second piece of fixation hardware is a second fastener.

23. The spinal stabilization device of claim 22, wherein the first arcuate arm is pivotably connected to the first fastener with a first pivot connection, and wherein the second arcuate arm is pivotably connected to the second fastener with a second pivot connection.

24. The spinal stabilization device of claim 23, wherein when installed within a patient's body the first arcuate arm remains free to pivot relative to the first fastener at the first pivot connection and the second arcuate arm remains free to pivot relative to the second fastener at the second pivot connection.

25. The spinal stabilization device of claim 22, wherein first arcuate arm is rigidly secured to the first fastener, and the second arcuate arm is rigidly secured to the second fastener.

26. The spinal stabilization device of claim 20, wherein the first piece of fixation hardware is an elongate rod attached to the first pedicle of the second vertebra with a pedicle screw, and the second piece of fixation hardware is an elongate rod attached to the second pedicle of the second vertebra with a pedicle screw.

27. The spinal stabilization device of claim 20, further including a fusion implant in conjunction with the dynamic stabilization device.

28. The spinal stabilization device of claim 20, further including a third vertebra located inferior to the second vertebra, an interdiscal space defined between the second vertebra and the third vertebra, further comprising a fusion implant located in the interdiscal space between the second vertebra and the third vertebra.

29. The spinal stabilization device of claim 28, wherein the dynamic stabilization device provides relief from adjacent level syndrome associated with fusing the second vertebra to the third vertebra.

30. The spinal stabilization device of claim 20, wherein the arcuate path of the first arcuate arm includes a compound curvature; and
wherein the arcuate path of the second arcuate arm includes a compound curvature.
